(19) United States
(12) Patent Application Publication
(10) Pub. No.: US 2004/0191852 A1
(43) Pub. Date: Sep. 30, 2004

(54) Methods for the identification of inhibitors of NADPH:protochlorophyllide oxidoreductase activity in plants

(76) Inventors: Keith Davis, Durham, NC (US); Adel Zayed, Durham, NC (US); Robert Ascenzi, Cary, NC (US); Douglas Boyes, Chapel Hill, NC (US); Rao Mulpuri, Apex, NC (US); Neil Hoffman, Chapel Hill, NC (US); Susanne Kjemtrup, Chapel Hill, NC (US); Carol Hamilton, Apex, NC (US); Jeffrey Woessner, Hillsborough, NC (US); Jorn Gorlach, Manchester, NJ (US); Lining Guo, Chapel Hill, NC (US); Veeresh Sevala, Cary, NC (US)

Correspondence Address: PARADIGM GENETICS, INC, 108 ALEXANDER DRIVE, P O BOX 14528, RTP, NC, 27709-4528, US

(21) Appl. No.: 10/401,048

(22) Filed: Mar. 27, 2003

(51) Int. Cl. C12Q 1/26

(52) U.S. Cl. 435/25

(57) ABSTRACT

The present inventors have discovered that NADPH:protochlorophyllide oxidoreductase (POR) is essential for plant growth. Specifically, the inhibition of POR gene expression in plant seedlings results in reduced growth and chlorosis. Thus, POR is useful as a target for the identification of herbicides. Accordingly, the present invention provides methods for the identification of herbicides by measuring the activity of a POR in the presence and absence of a compound, wherein an alteration of POR activity in the presence of the compound indicates the compound as a candidate for a herbicide.

### FIELD OF THE INVENTION

The invention relates generally to plant molecular biology. In particular, the invention relates to methods for the identification of herbicides.

### BACKGROUND OF THE INVENTION

NADPH:protochlorophyllide oxidoreductase (POR) catalyses the light-dependent reduction of protochlorophyllide (Pchlide) to chlorophyllide (Chlide), a key reaction in the chlorophyll biosynthesis pathway. In the presence of NADPH and light, the enzyme performs the trans-reduction of the double bond in ring D of the tetrapyrrol ring system (Martin G E et al. (1997) Biochem J. 325 (Pt 1):139-45.)

A. thaliana contains three POR isoforms, POR A, B and C. (Armstrong G A et al. (1995) Plant Physiol. 108(4):1505-17; Pattanayak G K and Tripathy B C. (2002) Biochem Biophys Res Commun. 291(4):921-4.) While POR A mRNA is only present in ethiolated seedlings, POR B mRNA is detected in dark-grown and in green seedlings (Holtorf H et al. (1995 ) Proc Natl Acad Sci USA. 92(8):3254-8). POR C is only found after illumination.

Angiosperms contain light-dependent POR (LPOR), whereas in gymnosperms, algae and photosynthetic bacteria a light-independent POR (DPOR) is found in addition. The DPOR consists of three different subunits and bears a high resemblance to nitrogenase.

The present invention discloses POR as a target for the evaluation of plant growth regulators, especially herbicide compounds, in plants.

### SUMMARY OF THE INVENTION

The present inventors have discovered that antisense expression of a POR cDNA in Arabidopsis causes chlorosis and reduced growth. Thus, the present inventors have discovered that POR is essential for normal plant development and growth, and is useful as a target for the identification of herbicides. Accordingly, in one embodiment the present invention provides methods for the identification of compounds that inhibit POR expression or activity, comprising: contacting a candidate compound with a POR and detecting the presence or absence of binding between the compound and the POR, wherein binding between the compound and the POR indicates the compound as a herbicide target. In another embodiment of the invention, methods are provided for the identification of compounds that inhibit POR enzyme activity, comprising: contacting a POR polypeptide with protochlorophyllide and NADPH in the presence and absence of a compound or contacting a POR polypeptide with chlorophyllide and NADP in the presence and absence of a compound; and determining a change in concentration for at least one of protochlorophyllide, NADPH, chlorophyllide, and/or NADP in the presence and absence of the compound, wherein a change in the concentration for any of protochlorophyllide, NADPH, chlorophyllide, and/or NADP indicates that the compound is a candidate herbicide.

### BRIEF DESCRIPTION OF THE FIGURE

FIG. 1 Diagram of the reversible reaction catalyzed by NADPH:protochlorophyllide oxidoreductase (POR). The enzyme catalyzes the reversible interconversion of protochlorophyllide and NADPH to chlorophyllide and NADP.

### DETAILED DESCRIPTION OF THE INVENTION

Definitions

The term “bDNA” refers to branched DNA.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions mediates the binding of two molecules to each other.

As used herein, the term “cDNA” means complementary deoxyribonucleic acid.

As used herein, the term “dI” means deionized.

As used herein, the term “ELISA” means enzyme-linked immunosorbent assay.

As used herein, the term “GUS” means β-glucouronidase.

The term “herbicide”, as used herein, refers to a compound useful for killing or suppressing the growth of at least one plant, plant cell, plant tissue or seed.

As used herein, the term “homologous POR” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence identity or each integer unit of sequence identity from 50-100% in ascending order to Arabidopsis POR protein (SEQ ID NO:2) and at least 10%, 25%, 50%, 75%, 80%, 90%, 95%, or 99% activity or each integer unit of activity from 10-100% in ascending order of the activity of Arabidopsis POR protein (SEQ ID NO:2). Examples of homologous POR's include, but are not limited to, POR A and POR C from Arabidopsis thaliana.

As used herein, the term “HPLC” means high pressure liquid chromatography.

The term “inhibitor,” as used herein, refers to a chemical substance that inactivates the enzymatic activity of POR or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide is “introduced” into a plant cell by any means, including transfection, transformation or transduction, electroporation, particle bombardment, agroinfection and the like. The introduced polynucleotide is maintained in the cell stably if it is incorporated into a non-chromosomal autonomous replicon or integrated into the plant chromosome. Alternatively, the introduced polynucleotide is present on an extra-chromosomal non-replicating vector and be transiently expressed or transiently active.

As used herein, the term “LB” means Luria-Bertani media.

As used herein, the terms “NADPH:protochlorophyllide oxidoreductase,” “NADPH:protochlorophyllide oxidoreductase polypeptide,” “POR”, and “POR polypeptide” refer to an enzyme that catalyzes the reversible interconversion of protochlorophyllide and NADPH to chlorophyllide and NADP.

As used herein, the term “Ni-NTA” refers to nickel sepharose.

As used herein, the term “PCR” means polymerase chain reaction.

The “percent (%) sequence identity” between two polynucleotide or two polypeptide sequences is determined according to the either the BLAST program (Basic Local Alignment Search Tool, Altschul and Gish (1996) Meth Enzymol 266: 460-480; Altschul (1990) J Mol Biol 215: 403-410) or using Smith Waterman Alignment (Smith and Waterman (1981) Adv Appl Math 2:482) using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant POR” is meant an enzyme found in at least one plant, and which catalyzes the reversible interconversion of protochlorophyllide and NADPH to chlorophyllide and NADP. The POR is from any plant, including monocots, dicots, C3 plants, and/or C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

As used herein, the term “SDS-PAGE” means sodium dodecyl sulfate-polyacrylimide gel electrophoresis.

The term “specific binding” refers to an interaction between POR and a molecule or compound, wherein the interaction is dependent upon the primary amino acid sequence or the conformation of POR.

The present inventors have discovered that inhibition of POR gene expression inhibits the growth and development of plant seedlings. Thus, the inventors are the first to demonstrate that POR is a useful target for the identification of herbicides.

Accordingly, the invention provides methods for identifying compounds that inhibit POR protein activity. Such methods include ligand binding assays, assays for enzyme activity and assays for POR gene expression. The compounds identified by the methods of the invention are useful as herbicides.

Thus, in one embodiment, the invention provides a method for identifying a compound as a candidate for a herbicide, comprising: contacting a POR with a compound; and detecting the presence and/or absence of binding between the compound and the POR, wherein binding indicates that the compound is a candidate for a herbicide.

By “POR” is meant an enzyme that catalyzes the reversible interconversion of protochlorophyllide and NADPH to chlorophyllide and NADP. In one embodiment of the invention, the POR has the amino acid sequence of a naturally occurring POR found in a plant, animal or microorganism. In another embodiment of the invention, the POR has an amino acid sequence derived from a naturally occurring sequence. In another embodiment the POR is a plant POR. Homologous POR's are useful in another embodiment of the invention.

One example of a cDNA encoding an Arabidopsis POR is set forth in SEQ ID NO:1 (TIGR database locus At21131/At4g27440). The POR polypeptide encoded by SEQ ID NO:1 is set forth in SEQ ID NO:2. DNA sequence encoding the POR N-terminal signal peptide is set forth in SEQ ID NO:3. The polypeptide encoded by SEQ ID NO:3 is set forth in SEQ ID NO:4. Nucleotide sequence of a truncated POR gene (tPOR) of 1008 nucleotides set forth in SEQ ID NO:5. The polypeptide encoded by SEQ ID NO:5 is set forth in SEQ ID NO:6. DNA encoding an N-terminal peptide fusion, provided by the pET30c (+) vector, that encodes a 6-His tag, thrombin cleavage site, S-tag, in that order, is set forth in SEQ ID NO:7. The polypeptide encoded by SEQ ID NO:7 is set forth in SEQ ID NO:8. DNA encoding a pET30c-tPOR fusion protein is set forth in SEQ ID NO:9. The polypeptide encoded by SEQ ID NO:9 is set forth in SEQ ID NO:10.

In one embodiment, the POR is an Arabidopsis POR. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii.

In various embodiments, the POR can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

POR polypeptides having at least 40% sequence identity with Arabidopsis POR (SEQ ID NO:2) protein are also useful in the methods of the invention. In one embodiment, the sequence identity is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence identity in ascending order with Arabidopsis POR (SEQ ID NO:2) protein. In addition, it is preferred that POR polypeptides of the invention have at least 10% of the activity of Arabidopsis POR (SEQ ID NO:2) protein. POR polypeptides of the invention have at least 10%, 15%, 20%, 25%, 30%, 35%, 40%, 45%, 50%, 55%, 60%, 65%, 70%, 75%, 80%, 85% or at least 90% of the activity of Arabidopsis POR (SEQ ID NO:2) protein.

Polypeptides consisting essentially of SEQ ID NO:2 are also useful in the methods of the invention. For the purposes of the present invention, a polypeptide consisting essentially of SEQ ID NO:2 has at least 90% sequence identity with Arabidopsis POR (SEQ ID NO:2) and at least 10% of the activity of SEQ ID NO:2. A polypeptide consisting essentially of SEQ ID NO:2 has at least 90%, 91%, 92%, 93%, 94%, 95%, 96%, 97%, 98%, or 99% sequence identity with SEQ ID NO:2 and at least 25%, 50%, 75%, or 90% of the activity of Arabidopsis POR (SEQ ID NO:2). Examples of polypeptides consisting essentially of SEQ ID NO:2 include, but are not limited to, polypeptides having the amino acid sequence of SEQ ID NO:2 with the exception that one or more of the amino acids are substituted with structurally similar amino acids providing a “conservative amino acid substitution.” Conservative amino acid substitutions are well known to those of skill in the art. Examples of polypeptides consisting essentially of SEQ ID NO:2 include polypeptides having 1, 2, or 3 conservative amino acid substitutions relative to SEQ ID NO:2.

Other examples of polypeptides consisting essentially of SEQ ID NO:2 include polypeptides having the sequence of SEQ ID NO:2, but with truncations at either or both the 3′ and the 5′ end. For example, polypeptides consisting essentially of SEQ ID NO:2 include polypeptides having 1, 2, or 3 amino acids residues removed from either or both 3′ and 5′ ends relative to SEQ ID NO:3. Additional examples of polypeptides consisting essentially of SEQ ID NO:2 are POR polypeptides in which the putative secretory leader sequence is absent, an example of which is the polypeptide of SEQ ID NO:6. In addition, POR polypeptides consisting essentially of SEQ ID NO:2 can be fusion proteins, such as SEQ ID NO: 10, in which a POR polypeptide is fused with another polypeptide or amino acid sequence to aid in secretion and/or purification as is known to those of skill in the art.

Fragments of a POR polypeptide are useful in the methods of the invention. In one embodiment, the POR fragments include an intact or nearly intact epitope that occurs on the biologically active wild-type POR. The fragments comprise at least 10 consecutive amino acids of a POR. The fragments comprise at least 15, 20, 25, 30, 35, 40, 50, 60, 70, 80, 90, 100, 125, 150, 175, 200, 225, 250, 275, 300, 325, 350, 357, 400 or at least 401 consecutive amino acids residues of a POR. In one embodiment, the fragment is from an Arabidopsis POR. In one embodiment, the fragment contains an amino acid sequence conserved among plant POR's.

Thus, in another embodiment, the invention provides a method for identifying a compound as a candidate for a herbicide, comprising: contacting a compound with a POR polypeptide selected from the group consisting of a POR polypeptide set forth in SEQ ID NO:2 or SEQ ID NO:10; a POR polypeptide consisting essentially of SEQ ID NO:2; a POR polypeptide comprising at least 10 consecutive amino acids of SEQ ID NO:2; and a POR polypeptide having at least 50% sequence identity with SEQ ID NO:2 and at least 10% of the activity of SEQ ID NO:2; and detecting the presence and/or absence of binding between the compound and the polypeptide, wherein binding indicates that the compound is a candidate for a herbicide.

Any technique for detecting the binding of a ligand to its target is useful in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a POR protein or a homologue, fragment or variant thereof, the unbound protein is removed and the bound POR is detected. In a preferred embodiment, bound POR is detected using a labeled binding partner, such as a labeled antibody. In a variation of this assay, POR is labeled prior to contacting the immobilized candidate ligands. Preferred labels include fluorescent or radioactive moieties. In other embodiments of the invention, detection methods include fluorescence correlation spectroscopy (FCS) and FCS-related confocal nanofluorimetric methods.

In another embodiment of the invention, compounds are tested as candidate herbicides based on ability to inhibit POR enzyme activity. The compounds are tested using either in vitro or cell based enzyme assays. Alternatively, compounds are tested by direct application to a plant or plant cell, or expressing it therein, and monitoring the plant or plant cell for changes or decreases in growth, development, viability or alterations in gene expression.

A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

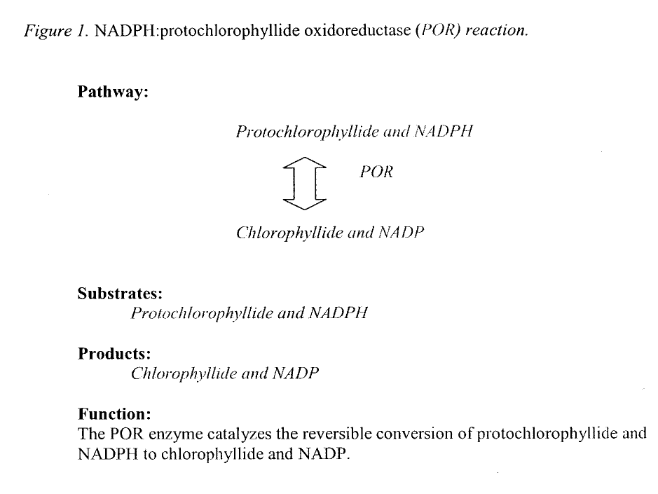

The ability of a compound to inhibit POR activity can be detected using in vitro enzymatic assays in which the disappearance of a substrate or the appearance of a product is directly or indirectly detected. POR catalyzes the reversible interconversion of protochlorophyllide and NADPH to chlorophyllide and NADP (see FIG. 1). Methods for measuring the progression of the POR enzymatic reaction and/or a change in the concentration of the individual reactants protochlorophyllide, NADPH, chlorophyllide and NADP, include spectrophotometry, fluorimetry, mass spectroscopy, thin layer chromatography (TLC) and reverse phase HPLC. In one embodiment, the reaction product chlorophyllide is directly measured with absorbance at 670 nm. In another embodiment, decrease of substrate and product formation are monitored by fluorescence measurements with excitation at 431 nm and emission at 628 nm and 665 nm. In another embodiment, due to the fact that the reaction is NADPH-dependent, NADPH degradation is quantified with resazurin dye. In another embodiment, a coupled assay is developed with chlorophyll synthase, which is an enzyme of the chlorophyll biosynthesis pathway. The POR reaction has a unique requirement for light. In one example, experiments with recombinant barley POR showed that after a single, saturating flash in the presence of excess enzyme, 5% Chlide of the total amount of Pchlide was formed in the system (Lebedev, N and Timko, M. P. (1999) Proc. Natl. Acad. Sci. USA 96, 9954-9959). The enzyme is capable of carrying out multiple turnovers, in contrast to the assumption that produced Chlide remains bound to POR A (Martin, G. E. M., Timko, M. P and Wilks, H. M. (1997) Biochem J. 325,139-145).

Thus, the invention provides a method for identifying a compound as a candidate herbicide, comprising: contacting protochlorophyllide and NADPH with a POR in the presence and absence of a compound or contacting chlorophyllide and NADP with a POR in the presence and absence of a compound; and determining a change in concentration for at least one of protochlorophyllide, NADPH, chlorophyllide and/or NADP in the presence and absence of the compound, wherein a change in the concentration for any of the above reactants indicates that the compound is a candidate for a herbicide. In one embodiment of the invention, the POR is the polypeptide set forth in SEQ ID NO:2. In another embodiment, the POR is the polypeptide set forth in SEQ ID NO:10. In another embodiment, the POR is a polypeptide consisting essentially of SEQ ID NO:2. In another embodiment, the POR is an Arabidopsis POR polypeptide. In another embodiment, the POR is a plant POR. In another embodiment the POR is a homologous POR.

Enzymatically active fragments of Arabidopsis POR set forth in SEQ ID NO:2 are also useful in the methods of the invention. For example, an enzymatically active polypeptide comprising at least 50 consecutive amino acid residues and at least 10% of the activity of Arabidopsis POR set forth in SEQ ID NO:2 are useful in the methods of the invention. In addition, enzymatically active polypeptides having at least 10% of the activity of SEQ ID NO:2 and at least 50%, 60%, 70%, 80%, 90%, 95%, 96%, 97%, 98% or 99% sequence identity with SEQ ID NO:2 are useful in the methods of the invention. Most preferably, the enzymatically active polypeptide has at least 50% sequence identity with SEQ ID NO:2 and at least 25%, 75% or at least 90% of the activity thereof.

Thus, the invention provides a method for identifying a compound as a candidate herbicide, comprising: contacting protochlorophyllide and NADPH or chlorophyllide and NADP with a polypeptide selected from the group consisting of: a polypeptide consisting essentially of SEQ ID NO:2, a polypeptide having at least 50% sequence identity with Arabidopsis POR set forth in SEQ ID NO:2 and having at least 10% of the activity thereof, a polypeptide comprising at least 50 consecutive amino acids of Arabidopsis POR set forth in SEQ ID NO:3 and having at least 10% of the activity thereof, and a polypeptide consisting of at least 50 amino acids and having at least 50% sequence identity with Arabidopsis POR set forth in SEQ ID NO:2 and having at least 10% of the activity thereof; contacting protochlorophyllide and NADPH or chlorophyllide and NADP with the polypeptide and a compound; and determining a change in concentration for at least one of protochlorophyllide, NADPH, chlorophyllide and/or NADP in the presence and absence of the compound, wherein a change in concentration for any of the above substances indicates that the compound is a candidate for a herbicide.

For the in vitro enzymatic assays, POR protein and derivatives thereof may be purified from a plant or may be recombinantly produced in and purified from a plant, bacteria or eukaryotic cell culture. Preferably POR proteins are produced using a baculovirus, E. coli or yeast expression system. Methods for purifying POR are found, for example, in Martin, G. E. M., Timko, M. P and Wilks, H. M. (1997) Biochem J. 325, 139-145; and Pattanayak, G. K. and Tripathy, B. C. (2002) Biochem Biophys Res Commun 291(4):921-4. Other methods for the purification of POR proteins and polypeptides are known to those skilled in the art.

As an alternative to in vitro assays, the invention also provides plant based assays. In one embodiment, the invention provides a method for identifying a compound as a candidate for a herbicide, comprising: a) measuring the expression or activity of a POR in a plant, or tissue thereof, in the absence of a compound; b) contacting the plant, or tissue thereof, with the compound and measuring the expression or activity of the POR in the plant, or tissue thereof; and c) comparing the expression or activity of the POR in steps (a) and (b), wherein an altered expression or activity in the presence of the compound indicates that the compound is a candidate for a herbicide. In one embodiment, the plant or tissue thereof is Arabidopsis thaliana.

In the methods of the invention, expression of a POR in a plant, or tissue thereof, is measured by detecting the POR primary transcript or mRNA, POR polypeptide or POR enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting POR RNA include, but are not limited to, amplification assays such as quantitative PCR, and/or hybridization assays such as Northern analysis, dot blots, slot blots, in-situ hybridization, transcriptional fusions using an POR promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system is useful to detect POR protein expression. For detection using gene reporter systems, a polynucleotide encoding a reporter protein is fused in frame with POR, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2: 1104; Prost et al. (1986) Gene 45: 107-111), β-galactosidase (Nolan et al. (1988) Proc Natl Acad Sci USA 85: 2603-2607), alkaline phosphatase (Berger et al. (1988) Gene 66: 10), luciferase (De Wet et al. (1987) Mol Cell Biol 7: 725-737), β-glucuronidase (GUS), fluorescent proteins, chromogenic proteins and the like. Methods for detecting POR activity are described above.

Chemicals, compounds, or compositions identified by the above methods as modulators of POR expression or activity are useful for controlling plant growth. For example, compounds that inhibit plant growth are applied to a plant or expressed in a plant to prevent plant growth. Thus, the invention provides a method for inhibiting plant growth, comprising contacting a plant with a compound identified by the methods of the invention as having herbicidal activity.

Herbicides and herbicide candidates identified by the methods of the invention are useful for controlling the growth of undesired plants, including including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants were grown in Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### Example 1

### Construction of a Transgenic Plant Expressing the Driver

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### Example 2

### Construction of POR Antisense Expression Cassettes in a Binary Vector

A fragment of the Arabidopsis thaliana cDNA corresponding to SEQ ID NO:1 was ligated into the PacI/AscI sites of an E. coli/Agrobacterium binary vector in the antisense orientation to yield an antisense expression cassette and a constitutive chemical resistance expression cassette located between right and left T-DNA borders. In this construct, transcription of the antisense RNA is controlled by an artificial promoter active only in the presence of the driver transcription factor described above. The artificial promoter contains four contiguous binding sites for the GAL4 transcriptional activator upstream of a minimal promoter comprising a TATA box. The ligated DNA was transformed into E. coli. Kanamycin resistant clones were selected and purified. DNA was isolated from each clone and characterized by PCR and sequence analysis confirming the presence of the antisense expression cassette.

### Example 3

### Transformation of Agrobacterium with the POR Antisense Expression Cassette

The binary vector described in Example 2 was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### Example 4

### Construction of Arabidopsis POR Antisense Target Plants

The POR antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4 ° C.

Transgenic Arabidopsis T1 seedlings were selected. Approximately 70 mg seeds from an agrotransformed plant were mixed approximately 4:1 with sand and placed in a 2 ml screw cap cryo vial. One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. The herbicide resistant plants represented stably transformed T1 plants.

### Example 5

### Effect of POR Antisense Expression in Arabidopsis Seedlings

The T1 POR antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. Antisense expression of the POR gene resulted in chlorosis and reduced growth in six of ten seedlings examined, indicating that the POR gene is essential gene for normal plant growth and development. Thus, the transgenic line containing the antisense construct for POR exhibited significant seedling abnormalities.

### Example 6

### Cloning, Expression, and Purification of the POR Protein

Cloning Strategy, Amino-Terminal Truncated Protochlorophyllide Oxidoreductase (tPOR):

Total RNA was collected from 14 day old Arabidopsis thaliana seedlings using published protocol and reagents (Trizol) from Life Technologies, Inc. (Rockville, Md.). 1 μg of total RNA was incubated with 10 pmol of custom oligo, AATTGCGGCCGCACTTCAAGTTTATTAGGC, in a reverse transcription reaction (Thermoscript RT kit, Life Technologies) according to the manufacturer's recommendations. Polymerase chain reaction (PCR) was carried out in a total volume of 50 μl with the following reagents: 2 μl of above RT reaction mixture, 20 mM Tris-HCl pH 8.8, 2 mM MgSO₄, 10 mM KCl, 10 mM (NH₄)₂SO₄, 0.1% Triton X-100, 0.1 mg/ml BSA, 10 mM dNTPs, 15 pmol of each primer (ATTGGTACCCAAACCGCTGCGACTTCA and AATTGCGGCCGCACTTCAAGTTTATTAGGC) and 2.5 units pfu Turbo polymerase (Stratagene, USA). PCR cycling was as follows: 94° C. (3 min), 55° C. (1 min), 68° C. (3 min) for 1 cycle, 94° C. (45 sec), 55° C. (30 sec), 68° C. (2 min) for 30 cycles, 68° C. (10 min). The resulting PCR product, and plasmid pET30c (+) (Novagen, Madison, Wis.), were digested with restriction endonucleases KpnI and NotI, as directed by the manufacturer (Life Technologies). Ligation of these two linear DNAs into the resulting recombinant clone pET30c-tPOR was accomplished by following instructions included with T4 DNA ligase (New England Biolabs). Integrity of the above clone was verified by DNA sequence analysis.

Methods Employed to Express the tPOR Gene:

Clone pET30c-tPOR was transformed into a proprietary bacterial strain, E. coli Rosetta (DE3) pLysS (Novagen), following the manufacture's instructions. Transformed bacteria were grown in LB liquid media (10 grams each tryptone and NaCl; 5 grams yeast extract; H₂O to one liter) supplemented with 34 micrograms/milliliter chloramphenicol and 50 micrograms/milliliter kanamycin, at 37° C. to an optical density of 0.6 at 600 nanometers. At this point, isopropylthio-Beta-galactoside (IPTG) was added to a final concentration of 1 mM and the culture was incubated at 23° C. for 16 additional hours. Bacteria were pelleted via centrifugation, the supernatant discarded, and the pellet frozen to −80° C.

Pellet (0.5 L) was resuspended in 15 ml lysis buffer: 50 mM Hepes, pH 7.5 containing 200 mM NaCl, benzonase (1 ul/2 ml), Protease inhibitor EDTA free-tablet (1 tablet/50 ml) and lysozyme (1 mg/ml), and sonicated on ice 6 times for 30 seconds each time. Sample was centrifuged at 15000×g for 10 minutes to sediment any insoluble material. Supernatant was recovered and used for purification of POR by Ni-NTA affinity chromatography (Qiagen).

The supernatant was applied to a 2 ml Ni-agarose column equilibrated with 50 mM Hepes, pH 7.5, containing 200 mM NaCl (Buffer A). The column was then washed with 15 ml of buffer A containing 20 mM imidazole and 5 ml of buffer A containing 50 mM imidazole. The bound POR was eluted with 5 ml of 500 mM imidazole in buffer A. Protein expression was confirmed by SDS-PAGE and Western blot analysis using anti-his antibody.

The enzyme was desalted using PD-10 gel filtration column (Amersham Pharmacia) in exchange buffer B (50 mM Hepes buffer , pH 7.5), containing of 10% glycerol and 1 mM DTT and protein was stored in 100 ul aliquots at −80° C. until use.

Protoclorophyllide Extraction and Purification:

Protochlorophyllide was isolated from Rhodobacter capsulatus ZY5. This strain is defective in BchL gene (Yang, Z. M. and Bauer, C. E. (1990) J Bacteriol 172:5001-10) which encodes for light independent protochlorophyllide oxidoreductase. Consequently monovinyl and divinyl protochlorophyllides accumulate in the bacterial cells under anaerobic (photosynthetic) growth conditions. R. capsulatus was first grown aerobically in PY medium (0.3% each Difco Bacto Peptone and Difco Yeast Extract containing 20 μg/ml kanamycin). Cells were then centrifuged and the pellet was transferred to modified RCV+ medium (Maleic acid 30 mM, (NH₄)₂ SO₄ 10 mM, KH₂PO₄ 10 mM, MgSO₄ 2 mM, CaCl₂ 1 mM, DMSO 0.5%, glucose 0.5%, pyruvate 0.5%, thiamin 15 μM) which was based on the recipe by Weaver, P. F, Wall, J. D. and Gest, H. (1975) Arch Microbiol 105:207-16. The medium was supplemented with the following trace elements: boron 100 μM, molybdenum 5 μM, copper 0.5 μM, zinc 2 μM, and manganese 20 μM and the pH was adjusted to 6.8. The cells were grown under low oxygen tension in the dark for 72 hours at 32° C. Cells were harvested by centrifugation and pigments were extracted by cold ethanol. The ethanol extract was partitioned between water-brine and diethyl ether. The ether layer was washed twice with water, then dried under a stream of argon to give partially purified pigment extract. Analysis of the purified pigments was performed by HPLC as well as by thin layer chromatography and product was confirmed by spectrofluorometry.

While the foregoing describes certain embodiments of the invention, it will be understood by those skilled in the art that variations and modifications may still fall within the scope of the invention.

What is claimed is:

1. A method for identifying a compound as a candidate for a herbicide, comprising:
a) contacting a POR polypeptide with a compound; and
b) detecting the presence or absence of binding between the compound and the POR polypeptide, wherein binding indicates that the compound is a candidate for a herbicide.

2. The method of claim 1, wherein the POR polypeptide is a plant POR polypeptide.

3. The method of claim 1, wherein the POR polypeptide is an Arabidopsis POR polypeptide.

4. The method of claim 1, wherein the POR polypeptide is SEQ ID NO:10.

5. A method for identifying a compound as a candidate for a herbicide, comprising:
a) contacting a compound with a polypeptide selected from the group consisting of:
i) a polypeptide consisting essentially of SEQ ID NO:2;
ii) a polypeptide having at least ten consecutive amino acids of SEQ ID NO:2;
iii) a polypeptide having at least 50% sequence identity with SEQ ID NO:2 and at least 10% of the activity of SEQ ID NO:2; and
iv) a polypeptide consisting of at least 50 amino acids having at least 50% sequence identity with SEQ ID NO:2 and at least 10% of the activity of SEQ ID NO:2; and
b) detecting the presence and/or absence of binding between the compound and the polypeptide, wherein binding indicates that the compound is a candidate for a herbicide.

6. A method for identifying a compound as a candidate for a herbicide, comprising:
a) contacting a POR polypeptide with protochlorophyllide and NADPH in the presence and absence of a compound or contacting a POR polypeptide with chlorophyllide and NADP in the presence and absence of a compound; and
b) determining a change in concentration for at least one of protochlorophyllide, NADPH, chlorophyllide and/or NADP in the presence and absence of the compound, wherein a change in the concentration for any of protochlorophyllide, NADPH, chlorophyllide and/or NADP indicates that the compound is a candidate for a herbicide.

7. The method of claim 6, wherein the POR is plant POR.

8. The method of claim 7, wherein the plant is a dicot.

9. The method of claim 7, wherein the plant is a monocot.

10. The method of claim 7, wherein the plant is other than a C3 plant.

11. The method of claim 7, wherein the plant is other than a C4 plant.

12. The method of claim 6, wherein the POR is an Arabidopsis POR.

13. The method of claim 6, wherein the POR is SEQ ID NO:10.

14. The method of claim 6, wherein the POR is a POR polypeptide consisting essentially of SEQ ID NO:2.

15. The method of claim 6, wherein the POR is a POR polypeptide selected from the group consisting of:
a) a polypeptide having at least 50% sequence identity with SEQ ID NO:2 and at least 10% of the activity of SEQ ID NO:2;
b) a polypeptide comprising at least 50 consecutive amino acids of SEQ ID NO:2 and having at least 10% of the activity of SEQ ID NO:2; and
c) a polypeptide consisting of at least 50 amino acids having at least 50% sequence identity with SEQ ID NO:2 and having at least 10% of the activity of SEQ ID NO:2.

16. A method for identifying a compound as a candidate for a herbicide, comprising:
a) measuring the expression of a POR in a plant, or tissue thereof, in the presence and absence of a compound; and
b) comparing the expression of the POR in the presence and absence of the compound, wherein an altered expression in the presence of the compound indicates that the compound is a candidate for a herbicide.

17. The method of claim 16, wherein the plant is Arabidopsis.

18. The method of claim 16, wherein the expression of the POR is measured by detecting the POR mRNA.

19. The method of claim 16, wherein the expression of the POR is measured by detecting the POR polypeptide.

20. The method of claim 16, wherein the expression of the POR is measured by detecting the POR polypeptide enzyme activity.

21. An isolated nucleic acid comprising a nucleotide sequence that encodes the polypeptide of SEQ ID NO:10.

22. An isolated nucleic acid comprising a nucleotide sequence that encodes a polypeptide consisting essentially of SEQ ID NO:2.

23. A recombinant polypeptide consisting essentially of the amino acid sequence of SEQ ID NO:2.

24. A recombinant polypeptide comprising the amino acid sequence of SEQ ID NO:10.

25. A method for identifying a compound as a candidate for a herbicide, comprising:
a) measuring the activity of a POR in the presence and absence of a compound, wherein an alteration of the POR activity in the presence of the compound indicates the compound as a candidate for a herbicide.

26. The method of claim 25, wherein the POR is plant POR.

27. The method of claim 26, wherein the plant is a dicot.

28. The method of claim 26, wherein the plant is a monocot.

29. The method of claim 26, wherein the plant is other than a C3 plant.

30. The method of claim 26, wherein the plant is other than a C4 plant.

31. The method of claim 25, wherein the POR is an Arabidopsis POR.

32. The method of claim 25, wherein the POR is SEQ ID NO:10.

33. The method of claim 25, wherein the POR is a POR polypeptide consisting essentially of SEQ ID NO:2.

34. The method of claim 25, wherein the POR is a POR polypeptide selected from the group consisting of:
a) a polypeptide having at least 50% sequence identity with SEQ ID NO:2 and at least 10% of the activity of SEQ ID NO:2;
b) a polypeptide comprising at least 50 consecutive amino acids of SEQ ID NO:2 and having at least 10% of the activity of SEQ ID NO:2; and
c) a polypeptide comprising at least 50 amino acids having at least 50% sequence identity with at least 50 consecutive amino acids of SEQ ID NO:2 and having at least 10% of the activity of SEQ ID NO:2.


SEQUENCE LISTING (13 sequences)

SEQ ID NO: 1 - DNA, 1206 bp, Arabidopsis thaliana
atggcccttcaagctgcttctttggtctcctctgctttctctgttcgcaaagatgcgaagttgaatgcttcttcatcatctttcaaggactcgagtctttttggtgcctccattaccgaccaaatcaaatccgaacatggatcttcctcgttaagattcaagagagaacagagcttaaggaatctagcaattcgagcccaaaccgctgcgacttcaagccctacagttacaaaatccgtggacggcaagaaaacgttgaggaaaggaaatgtggtggtcactggagcctcgtctgggttaggtctagccacggctaaagctctagctgagacagggaaatggaacgtgataatggcgtgcagagacttccttaaagccgagagagctgctaaatccgtagggatgcctaaagacagctacacagtgatgcatttagacttagcctcgttggacagcgtgagacagtttgttgataatttcaggagaacagagacgcctctcgatgttttggtctgcaatgctgcggtttatttcccgacagctaaagagcctacttacagtgctgaagggtttgagcttagtgttgcgacgaaccatttgggacattttcttctcgcaaggttgttgcttgatgacttgaagaaatctgattacccttcaaagcgtctcatcatcgtcggatccattaccgggaacacgaatacattggcgggtaatgtaccaccgaaggcgaatctcggtgatttgaggggtttagccggcggattaaacggtttaaacagctcagctatgattgatggaggagatttcgacggtgcaaaggcttacaaagacagtaaagtctgcaatatgttgacaatgcaagagtttcacaggcgtttccatgaagaaactggagtcactttcgcttcgctttaccccggttgcatcgcctccacaggtttattccgagagcacattcctctcttccgtgccctcttccctccctttcagaagtacatcactaaaggatatgtctccgaaacagagtcaggcaaaagacttgctcaggtggtgagtgatccaagcttgacgaaatcaggggtttattggagctggaacaatgcttcggcttcttttgagaaccagttatcagaagaagcaagtgacgttgagaaggctcgtaaagtgtgggagatcagtgagaagctcgtgggcttggcctaa

SEQ ID NO: 2 - protein, 401 aa, Arabidopsis thaliana
MALQAASLVSSAFSVRKDAKLNASSSSFKDSSLFGASITDQIKSEHGSSSLRFKREQSLRNLAIRAQTAATSSPTVTKSVDGKKTLRKGNVVVTGASSGLGLATAKALAETGKWNVIMACRDFLKAERAAKSVGMPKDSYTVMHLDLASLDSVRQFVDNFRRTETPLDVLVCNAAVYFPTAKEPTYSAEGFELSVATNHLGHFLLARLLLDDLKKSDYPSKRLIIVGSITGNTNTLAGNVPPKANLGDLRGLAGGLNGLNSSAMIDGGDFDGAKAYKDSKVCNMLTMQEFHRRFHEETGVTFASLYPGCIASTGLFREHIPLFRALFPPFQKYITKGYVSETESGKRLAQVVSDPSLTKSGVYWSWNNASASFENQLSEEASDVEKARKVWEISEKLVGLA

SEQ ID NO: 3 - DNA, 198 bp, Arabidopsis thaliana
atggcccttcaagctgcttctttggtctcctctgctttctctgttcgcaaagatgcgaagttgaatgcttcttcatcatctttcaaggactcgagtctttttggtgcctccattaccgaccaaatcaaatccgaacatggatcttcctcgttaagattcaagagagaacagagcttaaggaatctagcaattcgagcc

SEQ ID NO: 4 - protein, 66 aa, Arabidopsis thaliana
MALQAASLVSSAFSVRKDAKLNASSSSFKDSSLFGASITDQIKSEHGSSSLRFKREQSLRNLAIRA

SEQ ID NO: 5 - DNA, 1008 bp, Arabidopsis thaliana
caaaccgctgcgacttcaagccctacagttacaaaatccgtggacggcaagaaaacgttgaggaaaggaaatgtggtggtcactggagcctcgtctgggttaggtctagccacggctaaagctctagctgagacagggaaatggaacgtgataatggcgtgcagagacttccttaaagccgagagagctgctaaatccgtagggatgcctaaagacagctacacagtgatgcatttagacttagcctcgttggacagcgtgagacagtttgttgataatttcaggagaacagagacgcctctcgatgttttggtctgcaatgctgcggtttatttcccgacagctaaagagcctacttacagtgctgaagggtttgagcttagtgttgcgacgaaccatttgggacattttcttctcgcaaggttgttgcttgatgacttgaagaaatctgattacccttcaaagcgtctcatcatcgtcggatccattaccgggaacacgaatacattggcgggtaatgtaccaccgaaggcgaatctcggtgatttgaggggtttagccggcggattaaacggtttaaacagctcagctatgattgatggaggagatttcgacggtgcaaaggcttacaaagacagtaaagtctgcaatatgttgacaatgcaagagtttcacaggcgtttccatgaagaaactggagtcactttcgcttcgctttaccccggttgcatcgcctccacaggtttattccgagagcacattcctctcttccgtgccctcttccctccctttcagaagtacatcactaaaggatatgtctccgaaacagagtcaggcaaaagacttgctcaggtggtgagtgatccaagcttgacgaaatcaggggtttattggagctggaacaatgcttcggcttcttttgagaaccagttatcagaagaagcaagtgacgttgagaaggctcgtaaagtgtgggagatcagtgagaagctcgtgggcttggcctaa

SEQ ID NO: 6 - protein, 335 aa, Arabidopsis thaliana
QTAATSSPTVTKSVDGKKTLRKGNVVVTGASSGLGLATAKALAETGKWNVIMACRDFLKAERAAKSVGMPKDSYTVMHLDLASLDSVRQFVDNFRRTETPLDVLVCNAAVYFPTAKEPTYSAEGFELSVATNHLGHFLLARLLLDDLKKSDYPSKRLIIVGSITGNTNTLAGNVPPKANLGDLRGLAGGLNGLNSSAMIDGGDFDGAKAYKDSKVCNMLTMQEFHRRFHEETGVTFASLYPGCIASTGLFREHIPLFRALFPPFQKYITKGYVSETESGKRLAQVVSDPSLTKSGVYWSWNNASASFENQLSEEASDVEKARKVWEISEKLVGLA

SEQ ID NO: 7 - DNA, 114 bp, Artificial Sequence
  6-His tag thrombin cleavage S-tag n-terminal peptide provided by pET30c[+] vector (Novagen, Madison WI)
atgcaccatcatcatcatcattcttctggtctggtgccacgcggttctggtatgaaagaaaccgctgctgctaaattcgaacgccagcacatggacagcccagatctgggtacc

SEQ ID NO: 8 - protein, 38 aa, Artificial Sequence
  6-His tag thrombin cleavage S-tag protein N-terminal peptide provided by pET30[+] (Novagen, Madison, WI)
MHHHHHHSSGLVPRGSGMKETAAAKFERQHMDSPDLGT

SEQ ID NO: 9 - DNA, 1122 bp, Artificial Sequence
  Nucleotide sequence for fusion protein created by pET30[+] vector (Novagen, Madison, WI) and Arabidopsis POR protein
atgcaccatcatcatcatcattcttctggtctggtgccacgcggttctggtatgaaagaaaccgctgctgctaaattcgaacgccagcacatggacagcccagatctgggtacccaaaccgctgcgacttcaagccctacagttacaaaatccgtggacggcaagaaaacgttgaggaaaggaaatgtggtggtcactggagcctcgtctgggttaggtctagccacggctaaagctctagctgagacagggaaatggaacgtgataatggcgtgcagagacttccttaaagccgagagagctgctaaatccgtagggatgcctaaagacagctacacagtgatgcatttagacttagcctcgttggacagcgtgagacagtttgttgataatttcaggagaacagagacgcctctcgatgttttggtctgcaatgctgcggtttatttcccgacagctaaagagcctacttacagtgctgaagggtttgagcttagtgttgcgacgaaccatttgggacattttcttctcgcaaggttgttgcttgatgacttgaagaaatctgattacccttcaaagcgtctcatcatcgtcggatccattaccgggaacacgaatacattggcgggtaatgtaccaccgaaggcgaatctcggtgatttgaggggtttagccggcggattaaacggtttaaacagctcagctatgattgatggaggagatttcgacggtgcaaaggcttacaaagacagtaaagtctgcaatatgttgacaatgcaagagtttcacaggcgtttccatgaagaaactggagtcactttcgcttcgctttaccccggttgcatcgcctccacaggtttattccgagagcacattcctctcttccgtgccctcttccctccctttcagaagtacatcactaaaggatatgtctccgaaacagagtcaggcaaaagacttgctcaggtggtgagtgatccaagcttgacgaaatcaggggtttattggagctggaacaatgcttcggcttcttttgagaaccagttatcagaagaagcaagtgacgttgagaaggctcgtaaagtgtgggagatcagtgagaagctcgtgggcttggcctaa

SEQ ID NO: 10 - protein, 373 aa, Artificial Sequence
  pET30c-tPOR fusion protein from pET30c [+} vector (Novagen, Madison WI) and the POR protein from Arabidopsis
MHHHHHHSSGLVPRGSGMKETAAAKFERQHMDSPDLGTQTAATSSPTVTKSVDGKKTLRKGNVVVTGASSGLGLATAKALAETGKWNVIMACRDFLKAERAAKSVGMPKDSYTVMHLDLASLDSVRQFVDNFRRTETPLDVLVCNAAVYFPTAKEPTYSAEGFELSVATNHLGHFLLARLLLDDLKKSDYPSKRLIIVGSITGNTNTLAGNVPPKANLGDLRGLAGGLNGLNSSAMIDGGDFDGAKAYKDSKVCNMLTMQEFHRRFHEETGVTFASLYPGCIASTGLFREHIPLFRALFPPFQKYITKGYVSETESGKRLAQVVSDPSLTKSGVYWSWNNASASFENQLSEEASDVEKARKVWEISEKLVGLA

SEQ ID NO: 11 - DNA, 30 bp, Artificial Sequence
  Synthetic primer used for reverse transcription
aattgcggccgcacttcaagtttattaggc

SEQ ID NO: 12 - DNA, 27 bp, Artificial Sequence
  Synthetic primer used for PCR
attggtacccaaaccgctgcgacttca

SEQ ID NO: 13 - DNA, 30 bp, Artificial Sequence; identical to SEQ ID NO: 11
  Synthetic primer used for PCR